(19) United States
(12) Patent Application Publication
(10) Pub. No.: US 2009/0247882 A1
(43) Pub. Date: Oct. 1, 2009

(54) LIVING BODY HOLDING DEVICE

(75) Inventor: Yu TOJO, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/413,288

(22) Filed: Mar. 27, 2009

(30) Foreign Application Priority Data
Mar. 28, 2008 (JP) 2008-086130

(51) Int. Cl. A61B 6/00

(52) U.S. Cl. 600/476

(57) ABSTRACT

A concave accommodating section is formed inside a base body. The accommodating section has a shape substantially the same as the left half of a mouse. The mouse is put in the accommodating section in a lying posture with its left side down such that a surface wall of the accommodating section directly contacts with a body surface of the left half of the mouse. Thus, the mouse and its tumor are held in predetermined positions. The right half of the mouse including a body part of interest is exposed from the accommodating section.

### FIELD OF THE INVENTION

The present invention relates to a living body holding device for holding a living body while an image of fluorescence from this living body is captured.

### BACKGROUND OF THE INVENTION

An image taking apparatus with a light-proof housing is used in various fields. In such an image taking apparatus, a subject is placed in the housing and an image is taken by irradiating light to the subject from a light source provided in the housing. In the fields of biochemistry and molecular biology, chemiluminescent substances and fluorescent substances are used as marker substances. An imaging apparatus that takes images of light or fluorescence emitted from the above marker substance for evaluating gene sequences, gene expressions, isolation and identification of proteins, molecular weight and properties is known.

An image taking apparatus for performing so-called in vivo imaging is known (for example, see PCT International Publication No. WO 97/18841 corresponding to Japanese translation of PCT international application No. 2000-502884). In the in vivo imaging, genes, proteins, antibodies, or pharmacological substances marked with the above marker substance are given to a living body such as a mouse or a rat, and images of light from the marker substance distributed inside the living body or on the body surface are taken with a camera to examine gene expressions and pharmacological activities in the living body. For example, in a case that a fluorescent substance is used as a marker substance, excitation light is irradiated to the living body to excite the fluorescent substance. The camera receives the fluorescence caused by the excitation light and generates (takes) an image (hereinafter referred to as distribution image).

Since light such as fluorescence received by the camera is weak, a long exposure time is necessary for taking a distribution image. The living body is placed on an image taking stage in an anesthetized state to remain immobile during the exposure. However, the living body is soft and it may be difficult to keep the living body in a certain posture depending on a body part of interest. In such cases, the living body is inserted in, for example, a tube-like holding device so as to direct the body part of interest to the camera.

In a case that distribution images are taken at predetermined intervals to quantitatively analyze pharmacological activities or the like, it is necessary to hold a living body in a desired position every time an image is taken. However, it is difficult to hold the soft living body with the above tube-like holding device. The positional variations of the living body have caused errors and hindered a precise analysis.

To compare pharmacological activities, excitation light from an excitation light source of, for example, epi-illumination type, is concurrently irradiated to plural living bodies. In such a method, the irradiated light is reflected strongly off the surfaces of the living bodies, especially protrusions such as ears, to the camera. Such reflections cause noise in the distribution image. The excitation light source of the epi-illumination type may be disposed diagonally above the living body to avoid strong reflections to the camera. In this case, nonuniform irradiation is caused by irregular body surfaces such as wrinkles, and shadows overlapping with other living bodies.

### SUMMARY OF THE INVENTION

An object of the present invention is to provide a living body holding device capable of holding a living body in a desired posture easily.

Another object of the present invention is to provide a living body holding device to provide optimum irradiation conditions of excitation light.

In order to achieve the above objects and other objects, a living body holding device of the present invention includes a base body, an accommodating section and a light irradiation opening. The accommodating section is formed on the base body, and a surface wall of the accommodating section has a concave shape that fits to a shape of the living body. The surface wall exposes a part of a body surface of the living body to outside of the accommodating section. The surface wall makes direct contact with the rest of the body surface to hold the living body. The light irradiation opening is formed in the surface wall. The excitation light is irradiated through the light irradiation opening to the living body.

It is preferable that the base body is made of a light proof material that does not emit fluorescence by the excitation light.

It is preferable that the living body holding device includes a light source for irradiating the excitation light. The light source is provided inside the base body.

It is preferable that the living body holding device includes a light guide channel and a light guide. The light guide channel passes through the base body to the light irradiation opening. The light guide is inserted through the light guide channel. An end of the light guide is connected to an external light source device and the other end of the light guide extends to the light irradiation opening.

It is preferable that the living body holding device further includes a heater for heating the accommodating section so as to maintain the temperature of the living body.

It is preferable that the living body holding device further includes a size adjustment mechanism for adjusting a size of the accommodating section.

It is preferable that the living body holding device further includes a tilt adjustment mechanism for adjusting a tilt of the accommodating section.

According to the present invention, a living body such as a mouse or a rat is easily held in a desired posture. As a result, excitation light irradiates the living body uniformly without causing noise.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a perspective view of a living body holding device of the present invention;

FIG. 2 is a cross-sectional view of the living body holding device taken along a line II-II in FIG. 1;

FIG. 3 is an explanatory view of the living body holding device in which a mouse is held;

FIG. 4 is a schematic view of an image taking apparatus;

FIG. 5 is a cross-sectional view of an embodiment in which excitation light is irradiated to the mouse from an external light source device;

FIG. 6 is a plane view showing an embodiment in which an accommodating section is capable of adjusting its size;

FIG. 7 is a perspective view showing an embodiment in which the accommodating section is capable of adjusting its tilt; and

FIG. 8 is a partial cross-sectional view of a movable leg.

### DESCRIPTION OF THE PREFERRED EMBODIMENTS

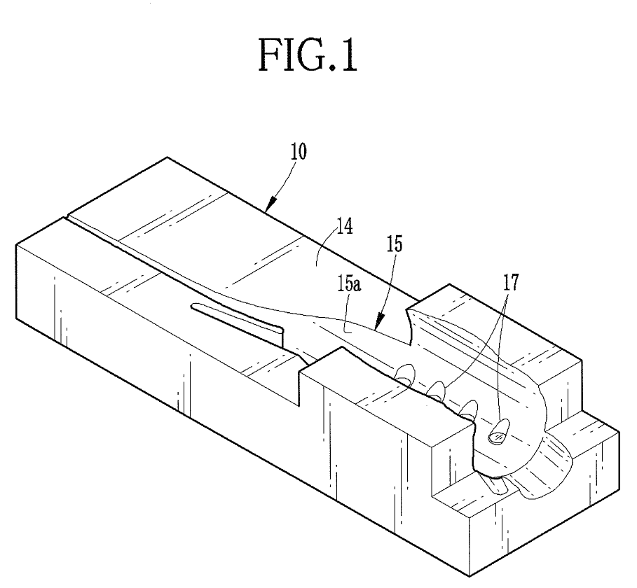

In FIG. 1, a living body holding device 10 of this embodiment holds a mouse 11 as an example of a living body in a lying posture with its left side down and its head to the right. The living body holding device 10 is used for taking images (hereinafter referred to as distribution images) of a fluorescent substance distributed on surfaces or in tissue of a shoulder 12a and a thigh 12b of a right lateral side 11a of the mouse 11, or in tissue of tumor and the like formed in those areas. Such fluorescent substance may be the fluorescent substance given to the mouse 11, tissue of a tumor that emits autofluorescence, or the like. Autofluorescence is fluorescence originating from substances other than the fluorescent substance of interest.

An accommodating section 15 is formed on an upper surface of a box-shaped base body 14 of the living body holding device 10. The accommodating section 15 is concave and substantially semi-circular in cross section into which the left half of the mouse 11 is fit from neck to tail. The accommodating section 15 may take any concave shape into which the mouse 11 of a standard size fits as long as the accommodating section 15 is capable of holding the mouse 11 in a desired posture.

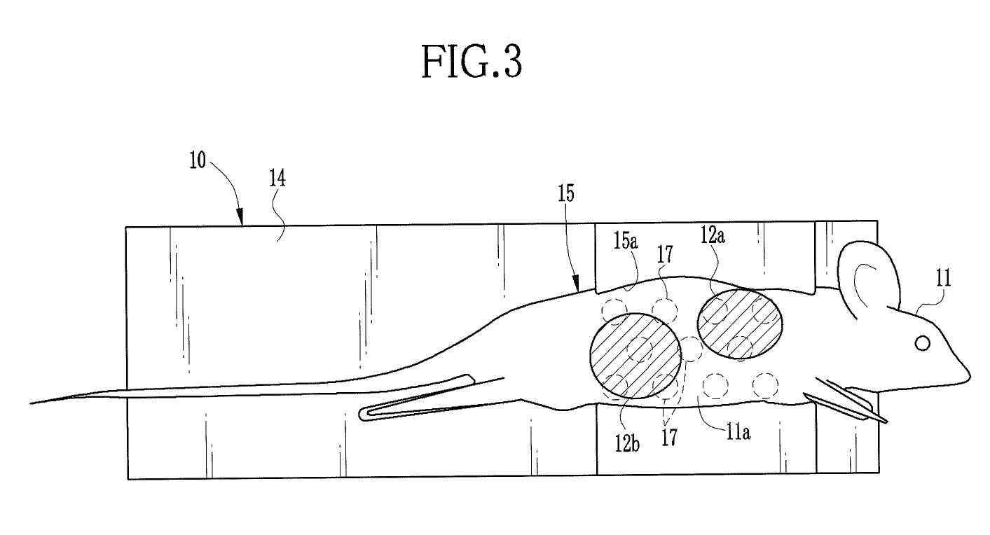

The mouse 11 is held in a lying posture by setting the mouse 11 in the accommodating section 15. A surface wall 15a has substantially the same contour as the left half of the body of the mouse 11. The left half of the body fits into and held by the surface wall 15a. Thus, the right half of the body including the right lateral side 11a (see FIG. 3) is exposed while a soft abdomen changeable in shape by gravity and a tumor inside the abdomen are held in predetermined positions. The right lateral side 11a is the body part of interest.

The abdomen of the mouse 11 easily changes its shape due to its weight. A protruded tumor tends to change its position due to its weight. In this embodiment, edge portions of the surface wall 15a are elongated around the right lateral side 11a so as to hold the right sides of the back and the abdomen of the mouse 11 securely. To put a mask for administering anesthetic gas on the nose of the mouse 11 easily, the base body 14 and the surface wall 15a extend to the neck of the mouse 11 such that the nose of the mouse 11 is projected out from the base body 14.

The above-described living body holding device 10 holds the mouse 11 from the neck to the tail. Alternatively, for example, a whole body, from the nose to the tip of the tail, or a part of the body, between forelegs and hind legs may be held. The size of the accommodating section 15 may differ from the size of the mouse 11 to some extent, because the body of the mouse 11 is very soft and easily changes its shape to fit into the surface wall 15a. This means that it is possible to deal with the mice 11 of different sizes only by preparing several living body holding devices 10 with stepwisely different sizes of accommodating sections 15.

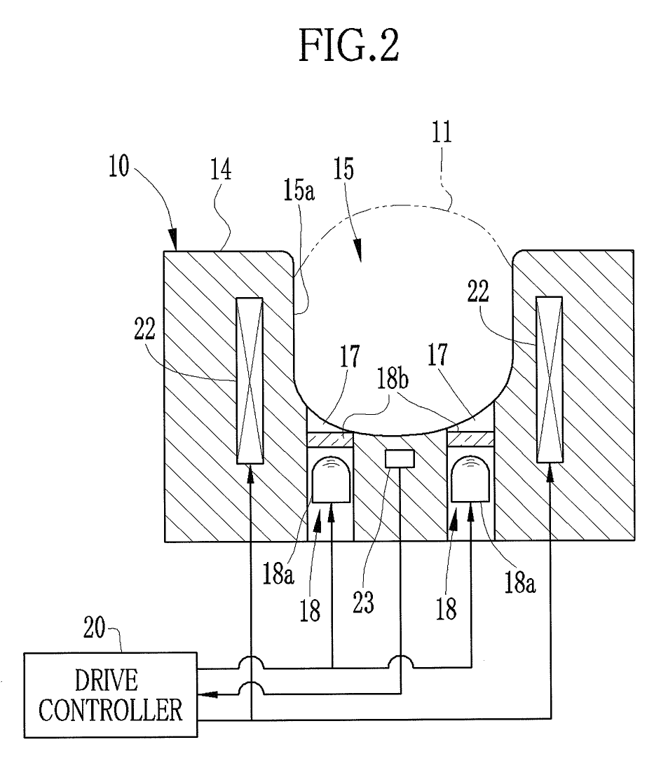

At least one light irradiation opening 17 is provided on the surface wall 15a that faces and directly contacts with the body surface opposite to that exposed outside. In other words, in this embodiment, the light irradiation opening 17 is provided on the surface wall 15a that faces and directly contacts with the left side of the body opposite to the right lateral side 11a. As shown in FIG. 3, plural light irradiation openings 17 are provided. As shown in FIG. 2, a light source 18 is disposed at the back of each light irradiation opening 17.

The light source 18 irradiates excitation light to the mouse 11. The excitation light excites the fluorescent substance distributed in the mouse 11 to cause fluorescence. The light source 18 includes a near-infrared LED 18a and a filter 18b. The filter 18b is a high pass filter that passes through light (near-infrared light), irradiated from the near-infrared LED 18a, in a wavelength range that excites the fluorescent substance to cause fluorescence, while filtering out the fluorescence from the fluorescent substance. As is well known, the wavelength of the fluorescence emitted from the fluorescent substance is shifted to a longer wavelength side relative to the wavelength of the excitation light due to a so-called Stokes shift. The structure of the light source 18 is an example. The light source 18 of any structure is used as long as it irradiates excitation light that excites the fluorescent substance.

When a drive controller 20 turns on the light source 18, the light source 18 irradiates excitation light through the light irradiation opening 17 to the mouse 11, and the excitation light is scattered inside the body of the mouse 11. The scattered excitation light irradiates the shoulder 12a, the thigh 12b, a tumor in the shoulder 12a and/or thigh 12b, and a tumor in other part of the body from many directions. As a result, the fluorescent substance is efficiently excited to fluoresce.

In this embodiment, eleven light irradiation openings 17 in a staggered arrangement are used for the mouse 11 of approximately 2 cm in width. The light source 18 is provided for each light irradiation opening 17. The number, size, and arrangement of the light irradiation openings 17 may be determined as necessary depending on required excitation light intensity, intensity of excitation light irradiated from each light irradiation opening 17, a body part of the mouse 11 to which excitation light is irradiated, or the like. Alternatively, only one light irradiation opening 17 may be used or plural light sources 18 may be used per light irradiation opening 17.

A camera filter is provided on the camera side to filter out the excitation light. Actually, however, the camera filter cannot filter out the excitation light completely. When strong excitation light leaks from the living body holding device 10 and is reflected off the protrusions of the body of the mouse 11 such as ears to the camera 33, the reflected excitation light causes noise in the distribution image. For this reason, it is preferred not to dispose the light irradiation opening 17 in a position where the light irradiation opening 17 and the mouse 11 are in poor contact. It is preferred to select a position where the excitation light hardly leaks even if the body of the mouse 11 is not in tight contact with the surface wall 15a. Therefore, it is most preferable to dispose the light irradiation opening 17 to face the body surface opposite to the exposed body surface of interest as described above.

As shown in FIG. 2, a heater 22 and a temperature sensor 23 are provided inside the base body 14. The heater 22 heats the surface wall 15a to maintain the body temperature of the mouse 11 with the heat conducted through the surface wall 15a. The temperature sensor 23 detects a temperature of the base body 14. The drive controller 20 turns the heater 22 on or off based on the detection results of the temperature sensor 23 to keep the temperature of the surface wall 15a equal to the body temperature of the mouse 11. Thus, the body temperature of the mouse 11 is kept at a certain value and a drop in the body temperature under anesthesia is prevented.

The base body 14 is made of a material that does not emit autofluorescence to prevent noise in the distribution image. In addition, to heat the mouse 11 by the heater 22 efficiently, the base body 14 is made of a material with high thermal conductivity. Considering the above, metals are preferable as the material of the base body 14. Alternatively, plastic polymers which do not emit autofluorescence may be used. In this embodiment, the base body 14 is made of aluminum that does not emit autofluorescence and has high thermal conductivity.

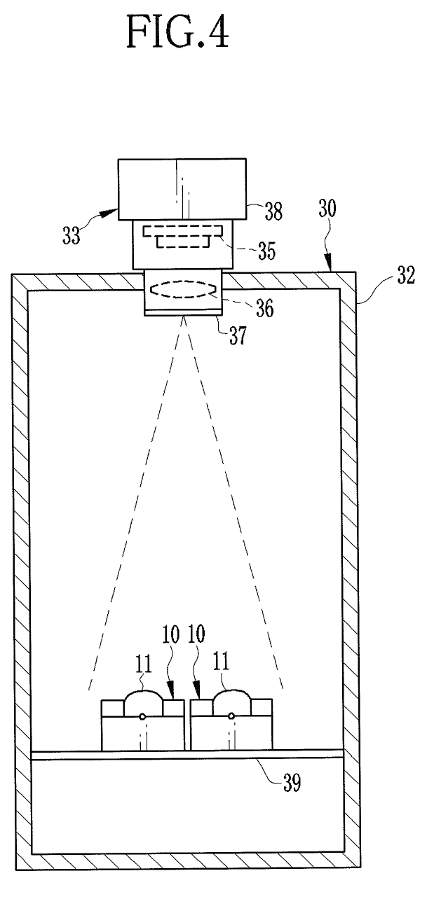

In FIG. 4, an image taking apparatus 30 is provided with an image taking chamber 32. The image taking chamber 32 is provided with a lid (not shown), and opened and closed with the lid. In an upper portion through the image taking chamber 32, a camera 33 is provided. The lid is opened to put the living body holding device 10 in or remove it from the image taking chamber 32. The image taking chamber 32 becomes lightproof when the lid is closed. Any light other than fluorescence emitted from the mouse 11 is prevented from entering the camera 33.

The camera 33 provided in the upper portion through the image taking chamber 32 includes an image sensor 35, a taking lens 36, a camera filter 37 and a cooler 38. The taking lens 36 and the camera filter 37 are inside the image taking chamber 32. The image sensor 35 is the same as that used in a digital camera or the like. A plurality of light receiving elements are arranged in a matrix on a light receiving surface. The distribution image is taken with the image sensor 35 by receiving fluorescence emitted from the fluorescent substance distributed in the mouse 11 with a long exposure time of, for example, 10 seconds. During the exposure, the cooler 38 cools the image sensor 35 to reduce a dark current, preventing noise in the distribution image.

The taking lens 36 forms an image of the fluorescence from the mouse 11 on the image sensor 35. The camera filter 37 filters out excitation light and passes through fluorescence from the fluorescent substance distributed in the mouse 11. The camera filter 37 is a low pass filter that filters out excitation light, in other words, for example, light with a wavelength shorter than that of the fluorescence from the fluorescent substance.

An image taking stage 39 is provided directly below the taking lens 36. The living body holding device 10 accommodating the mouse 11 is placed on the image taking stage 39. To take an image of mice 11 in one frame, the living body holding devices 10 each accommodating the mouse 11 are placed on the image taking stage 39. In FIG. 4, two living body holding devices 10 are illustrated as an example.

Next, an operation of the above embodiment is described. The mouse 11 to which the fluorescent substance is given in advance undergoes preliminary anesthesia. The anesthetized mouse 11 is accommodated in the accommodating section 15 such that the mouse 11 fits into the accommodating section 15. Thus, the mouse 11 is held in a desired posture. Since the left half of the body of the mouse 11 is supported by the surface wall 15a, the mouse 11 is kept in a predetermined shape. The right lateral side 11a of the mouse 11 and tumor tissue in the right lateral side 11a are kept in fixed positions.

As described above, the body, and the body part and tissue of interest are held in the same position every time the mouse 11 is held in the living body holding device 10. In a case that a plurality of mice 11 are held in separate living body holding devices 10, each mouse 11 is held in the same manner.

After the mouse 11 is held in the living body holding device 10, the living body holding device 10 is disposed on the image taking stage 39 of the image taking apparatus 30. One or more anesthetic masks (not shown) are provided in the image taking chamber 32. The nose of the mouse 11 is covered with the anesthetic mask, and anesthetic gas is administered to keep the mouse 11 under anesthesia. To take an image of mice 11 at a time, each living body holding device 10 accommodating the mouse 11 is disposed inside the image taking chamber 32 in the same manner as above with the nose of each mouse 11 covered with the anesthetic mask.

Thereafter, the drive controller 20 is operated to turn on the light sources 18. After the heating by the heater 22 is started, the lid of the image taking apparatus 30 is closed. Then, the image taking apparatus 30 is operated to turn on the camera 33, and exposure of the distribution image is started. The living body holding device 10 may be heated by the heater 22 before accommodating the mouse 11 in the accommodating section 15. The drive controller 20 may be provided in the image taking apparatus 30 so as to turn on/off the light sources 18 and start heating by the heater 22 with the operation of the image taking apparatus 30.

Upon turning on the light sources 18, excitation light is irradiated through the light irradiation openings 17 to the mouse 11. The excitation light scatters inside the body of the mouse 11 and irradiates the fluorescent substance distributed on or inside the body of the mouse 11, thereby exciting the fluorescent substance. The image sensor 35 receives the fluorescence emitted from the fluorescent substance through the camera filter 37 and the taking lens 36. When imaging is started, light receiving elements converts the received fluorescence into charge and the charge is stored. Thus, the charge is gradually stored in the image sensor 35 with time and the distribution image is generated. The distribution image is read from the image sensor 35 after a predetermined lapse of time, and stored in a memory. Thereafter, the distribution image is displayed on a monitor (not shown).

Since the light irradiation openings 17 are in direct contact with the body surface of the mouse 11, the excitation light is irradiated to the mouse 11 efficiently, and leakage of the excitation light is prevented. Naturally, the excitation light is prevented from being reflected from projected body parts such as ears. Accordingly, the distribution image is taken without noise. Even if plural living body holding devices 10 are disposed close to each other, excitation light from the light source 18 of the living body holding device 10 does not leak to adjacent living body holding devices 10. Accordingly, the distribution images are taken without variations in excitation light intensity. Thus, the distribution image is taken properly while the mouse 11, the body part of interest, and tumor tissue formed therein are held in desired positions.

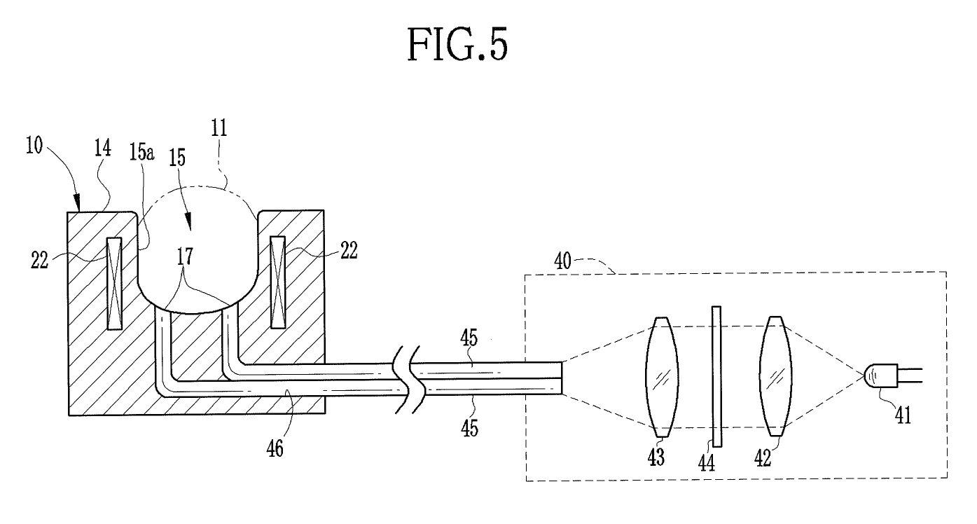

In the above embodiment, the excitation light source is incorporated in the living body holding device 10. Alternatively, a light source device may be provided separately from the living body holding device 10. In FIG. 5, a component similar to that in the first embodiment is designated by the same numeral as in the first embodiment, and a description thereof is omitted.

An external light source device 40 includes a light source 41 such as a near-infrared LED or the like, a pair of lenses 42 and 43, and a filter 44. Light irradiated from the light source 41 becomes parallel luminous flux through the lens 42 and enters the filter 44 disposed between the lenses 42 and 43. The filter 44 passes light with a wavelength of excitation light that excites the fluorescent substance to fluoresce. Conversely, the filter 44 filters out light with a wavelength of the fluorescence from the fluorescent substance. The excitation light passed through the filter 44 is gathered at ends of optical fibers (light guides) 45 by the lens 43 and enters the light guides 45.

Light guide channels 46 are formed in the base body 14. Each of the other ends of the light guides 45 is connected to the light irradiation opening 17 through the light guide channel 46. Thus, the light from the external light source device 40 is irradiated through the light irradiation openings 17 to the mouse 11. Alternatively, the other ends of the light guides 45 may be in direct contact with the mouse 11.

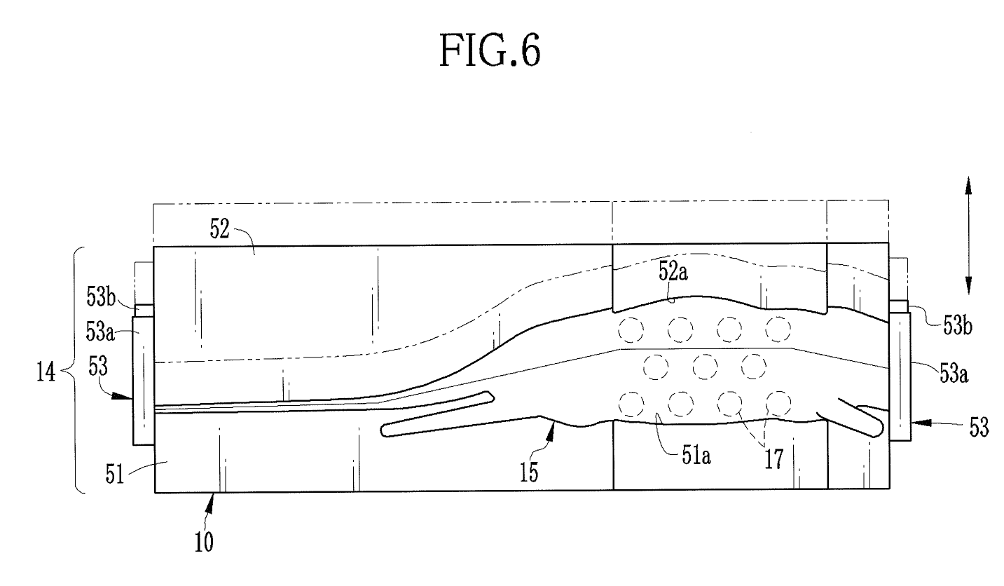

The living body holding device 10 may have a size adjustment mechanism. In FIG. 6, a component substantially the same as that in FIG. 1 is designated by the same numeral as in FIG. 1, and a description thereof is omitted.

The base body 14 includes a first slide unit 51 and a second slide unit 52. The first slide unit 51 is formed with a surface wall 51a that comes in direct contact with the abdominal surface of the mouse 11. The second slide unit 52 is formed with a surface wall 52a that comes in direct contact with the dorsal surface of the mouse 11. The surface walls 51a and 52a form the accommodating section 15.

The second slide unit 52 is slidable relative to the first slide unit 51, and slides between a minimum size position and a maximum size position. In FIG. 6, the minimum size position is shown in solid lines and the maximum size position is shown in chain double dashed lines. The minimum size position makes a space between the opposing surface walls 51a and 52a minimum. The maximum size position makes a space between the opposing surface walls 51a and 52a maximum.

For example, a slidable plate member (not shown) is disposed below the first slide unit 51. The plate member is pulled out from the first slide unit 51 along with sliding of the second slide unit 52. Thus, there is no gap between the first and second slide units 51 and 52 while the second slide unit 52 is slid from the minimum size position to the maximum size position, thereby preventing the mouse 11 from being caught in the gap between the first and second slide units 51 and 52.

A slide mechanism 53 is attached to the first slide unit 51 and the second slide unit 52. The slide mechanism 53 guides the second slide unit 52 to slide between the minimum size position and the maximum size position. The slide mechanism 53 includes, for example, a curved (tube-like) stationary section 53a and a movable section 53b. The stationary section 53a is fixed to the first slide unit 51. The movable section 53b is fixed to the second slide unit 52 and fits with the stationary section 53a in a slidable manner. In this embodiment, a size adjustment mechanism for adjusting the size of the accommodating section 15 includes the first slide unit 51, the second slide unit 52 that is slidable relative to the first slide unit 51, and the slide mechanism 53.

As described above, the size of the accommodating section 15 is adjusted by sliding the second slide unit 52 to fit with the size of the mouse 11 while the surface walls 51a and 52a are in direct contact with the mouse. Thus, the mouse 11 is properly held and kept in the desired posture.

In the above embodiment, the size of the accommodating section is adjusted in a body height direction. Alternatively, the size of the accommodating section may be adjusted in a body length direction, a depth direction, or a combination of them. It is possible to make the size of a part of the accommodating section adjustable, for example, a part accommodating the body of the mouse. Various adjustment methods can be adopted to change the size of the accommodating section. For example, a balloon may be provided on a part of the surface wall of the accommodating section. After the mouse is accommodated, the balloon is inflated with appropriate pressure to be in direct contact with the mouse. In this case, the balloon is made of a material that shields excitation light and does not emit autofluorescence. Alternatively, a liquid that shields excitation light may be put in the balloon.

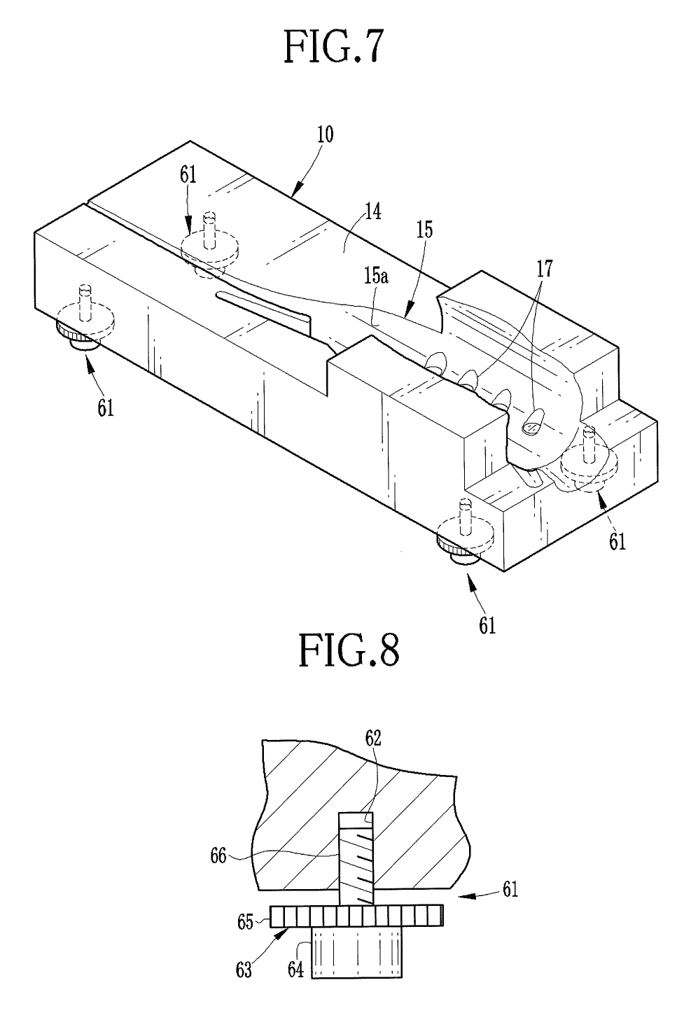

Another embodiment is shown in FIG. 7. In FIG. 7, a component substantially the same as that in the embodiment shown in FIG. 1 is designated by the same numeral as in the FIG. 1, and a description thereof is omitted.

In this embodiment, the living body holding device 10 is provided with a movable leg 61 on each corner of a bottom portion of the base body 14. The four movable legs 61 constitute a tilt adjustment mechanism for adjusting a tilt of the accommodating section 15. As shown in FIG. 8, each movable leg 61 includes a mounting foot 63 and a screw hole 62 formed in the bottom portion of the base body 14. The mounting foot 63 includes a foot body 64 that contacts with a mounting plane such as the image taking stage 39, a dial 65 formed integrally with the foot body 64, and a screw 66 that is screwed in the screw hole 62.

With the above structure, a projected length of the mounting foot 63 from the base body 14 is changed by rotating the dial 65. The tilt of the base body 14, namely, the tilt of the accommodating section 15 is adjusted as desired by changing the projected length of each mounting foot 63. After the mouse 11 is accommodated in the accommodating section 15, the tilt of the accommodating section 15 is changed easily with the operation of the dial 65 to direct the body part of interest to the camera 33, for example.

The tilt adjustment mechanism described above is a mere example, and not limited the above. The tilt adjustment mechanism may be combined with the size adjustment mechanism shown in FIG. 6.

In the above embodiments, the mouse is described as an example of the living body to be held in the living body holding device. The present invention is also applicable to other living bodies such as dogs and cats. The shape of the accommodating section may be properly changed depending on the living body used and the body part of interest. A living body holding device without the light irradiation opening is useful in a case that excitation light is irradiated from outside or irradiation of excitation light is unnecessary.

Various changes and modifications are possible in the present invention and may be understood to be within the present invention.

What is claimed is:

1. A living body holding device for holding a living body while an image is generated by a camera receiving fluorescence from said living body, said fluorescence being caused by irradiating excitation light to said living body, said living body holding device comprising:
a base body;
an accommodating section formed on said base body, said accommodating section having a surface wall in a concave shape that fits to the shape of said living body, said surface wall exposing a part of a body surface of said living body to outside of said accommodating section, while making direct contact with the rest of said body surface to hold said living body; and
a light irradiation opening formed in said surface wall, said excitation light being irradiated through said light irradiation opening to said living body.

2. The living body holding device of claim 1, wherein said base body is made of a lightproof material that does not emit fluorescence by said excitation light.

3. The living body holding device of claim 1 further including a light source for irradiating said excitation light, said light source being provided inside said base body.

4. The living body holding device of claim 1 further including:
a light guide channel passing through said base body to said light irradiation opening; and
a light guide inserted through said light guide channel, an end of said light guide being connected to an external light source device and the other end of said light guide extending to said light irradiation opening.

5. The living body holding device of claim 1 further including a heater for heating said accommodating section so as to maintain the temperature of said living body in said accommodating section.

6. The living body holding device of claim 1 further including a size adjustment mechanism for adjusting a size of said accommodating section.

7. The living body holding device of claim 1 further including a tilt adjustment mechanism for adjusting a tilt of said accommodating section.
